(19) United States
(12) Patent Application Publication
(10) Pub. No.: US 2006/0111677 A1
(43) Pub. Date: May 25, 2006

(54) Protective sleeve for a removable tip syringe

(75) Inventor: Lanny Nesbitt, Strasburg, PA (US)

Correspondence Address: DENTSPLY INTERNATIONAL INC, 570 WEST COLLEGE AVENUE, YORK, PA, 17404, US

(73) Assignee: Dentsply Research and Development Corp., Los Angeles, CA (US)

(21) Appl. No.: 11/319,979

(22) Filed: Dec. 28, 2005

Related U.S. Application Data
(63) Continuation of application No. 10/045,118, filed on Nov. 1, 2001, now abandoned.
(60) Provisional application No. 60/248,408, filed on Nov. 14, 2000.

(51) Int. Cl. A61M 5/32; A61M 5/00; A61M 31/00

(52) U.S. Cl. 604/192; 604/263; 604/506

(57) ABSTRACT

A syringe sleeve (10) receives a syringe (11) without the syringe (10) dispensing tip (12). After the syringe (11) is received within the sleeve (10), the dispensing tip (12) is affixed to the syringe (11). This causes the end (50) of the sleeve (10) to open and the end (50) to be physically impinged between the tip (12) and the syringe (11). The syringe (11) is thus protected from the operative environment.

### RELATED APPLICATIONS

This application is a Continuation application of U.S. patent application Ser. No. 10/045,118 (Case DPD-7281) filed Nov. 1, 2001, which claims the benefit from Provisional Application No. 60/248,408 filed Nov. 14, 2000.

### TECHNICAL FIELD

The present invention is a protective sleeve for a syringe. More particularly, a syringe may be inserted into the inventive sleeve. The sleeve is of a flexible plastic material. The syringe without its dispensing tip is inserted into the sleeve and then the tip is placed onto the syringe.

### BACKGROUND OF THE INVENTION

Protective coverings for syringes, such as dental syringes, are known in the art. For example, U.S. Pat. No. 5,332,092 discloses a protective syringe sheath. This sheath is useful with a material dispensing syringe wherein the syringe is inserted into the sheath. The dispensing tip is caused to pierce the end of the sheath or to pass through an orifice in the end. By being tightly held to the body of the syringe, the syringe is at least partially protected from the environment. In dental use, the dispensing tip can be replaced and the syringe inserted into a new sheath, and the syringe can be used with a new patient. These types of syringes often are preloaded with enough material to accomplish several or more procedures on different patients.

The protruding or piercing type sheath of the '092 patent continue to cause some concern of cross contamination between patients. While the '092 style sheath offers some protection, there is still an opening between the environment (the oral cavity for example) and the syringe itself. The '092 patent discloses minimizing this possibility by making the sheath fit tightly to the body of the syringe. T is necessary of course, to provide a sheath tailor-made to the dimensions of the syringe with which it will be employed. Otherwise, the protective aspects of the sheath are eliminated.

A need exists for a protective sleeve for a replaceable type, pre-loaded syringe that will further minimize the risk of contamination of the syringe itself. It would also be beneficial, and encourage more widespread use, if the protective sleeve was useful with a wider range of syringe dimensions than being limited to substantially one size.

### SUMMARY OF THE INVENTION

It is therefore, an object of the invention to provide a protective sleeve for a replaceable tip type syringe.

It is another object of the invention to provide a sleeve that offers improved contamination protection to the syringe.

It is a further object of the invention to provide a sleeve that is useful with a range of syringes having differing dimensions.

These and other object of the invention, which will become apparent from the following disclosure, are accomplished by the invention as hereinafter described.

In general, a protective sleeve is useful with a replaceable tip style syringe. The sleeve has an elongated body with a syringe receiving opening at one end, and a syringe engaging end at the opposite end of the elongated body. The syringe, without the its dispensing tip in place, is inserted into the protective sleeve y being passed through the syringe receiving opening. The tip is then placed onto the syringe, physically trapping the flexible sleeve therebetween. In this manner, there is virtually no open passage between the environment and the syringe inside the sleeve.

### BRIEF DESCRIPTION OF THE DRAWINGS

FIG. 1 is perspective view of the protective sleeve according to the present invention, shown together with a syringe and a separated dispensing tip outside of the sleeve.

FIG. 2 is a perspective view of the sleeve of FIG. 1, showing the syringe in place inside of the sleeve and the tip outside of the sleeve.

FIG. 3 is a perspective view as in FIG. 2, showing the direction of placement of the tip upon the syringe.

FIG. 4 is a perspective view as in FIG. 3, showing the dispensing tip in place upon the syringe, with the sleeve impinged therebetween.

FIG. 5 is a side elevational and cross sectional view taken along line A-A of FIG. 3.

FIG. 6 is a side elevational and cross sectional view taken along line B-B of FIG. 4, and showing the placement of the dispensing tip upon the syringe held within the sleeve.

### PREFERRED EMBODIMENTS FOR CARRYING OUT THE INVENTION

There is shown by way of example, a syringe sleeve by the number 10 upon the attached drawings, embodying the concepts of the present invention. Syringe sleeve 10 can be used with any syringe, but is preferably used with a replaceable tip syringe 11 that uses a replaceable dispensing tip 12. Such syringes and dispensing tips are well known in the art, and often are marketed preloaded with a certain material, such as a dental resin, sealant or the like. For contamination prevention purposes, these syringes are normally supplied with disposable and/or replaceable dispensing tips. Such tips may be placed onto the syringe by any conventional mean, including threads, bayonet lugs, or the like. One useful and common connection between the dispensing tip and the syringe is depicted in the drawings as a leur connector, it being understood that any connection is useful and is within the scope of the invention.

As such, tip 12 has a dispensing proboscis 13 and a connector body 14 with a thread engaging flange 15. Connector body 14 is generally hollow and is in fluid communication with hollow proboscis 13. The syringe 11 is provided with a conventional hollow barrel 20 and an inserted and axially displaceable plunger 21. At dispensing end 22 of syringe 11 is a tip connector assembly 23. Connector assembly 23 has means to permit physical engagement and coupling with tip 12, such as threads 30 contained within leur connector barrel 31. Syringe 11 also has dispensing protrusion 32 in fluid communication with the interior of barrel 20. As plunger 21 is displaced toward dispensing end 22, material (not shown) within barrel 20 is caused to move through dispensing protrusion 32 which is preferably, at least partially surrounded by connector barrel 30 of connector assembly 23.

Sleeve 10 is preferably made from a flexible plastic material, such as polyethylene, polypropylene, or any other material that can be made thin and flexible, and which will not readily break down in the intended environment.

Sleeve 10 is preferably configured to have an elongated, hollow interior body 40, with an open end 41. Open end 41 is configured to permit a syringe such as syringe 11 into the hollow interior of sleeve 11 through open end 41. More preferably, sleeve 10 is configured to have a length greater than the length of the intended syringe 11. Sleeve 10 may also be provided with an extending section 42. After syringe 11 is placed into sleeve 10, as shown in FIG. 4, extending section 42 can be folded over onto or along body 40, thus effectively closing off open end 41, and further protecting the then enclosed syringe 11 from the environment.

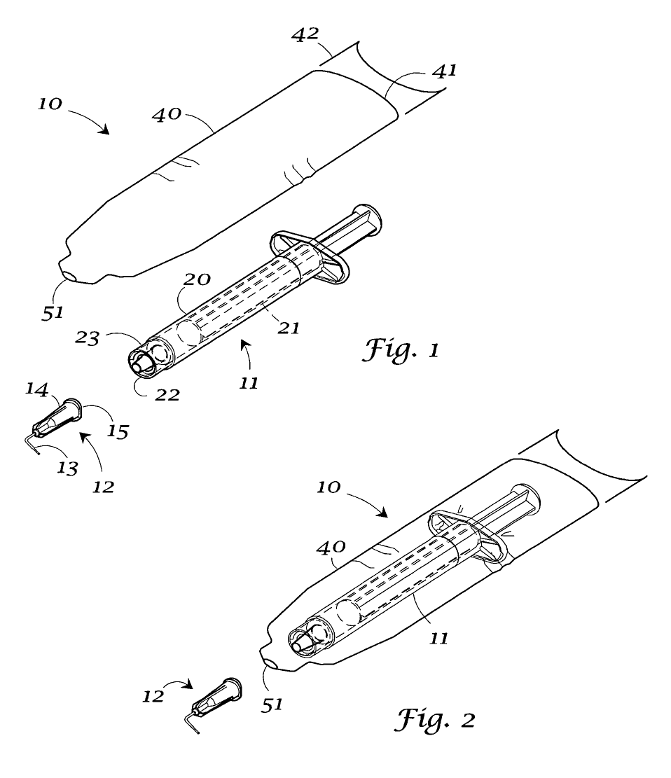

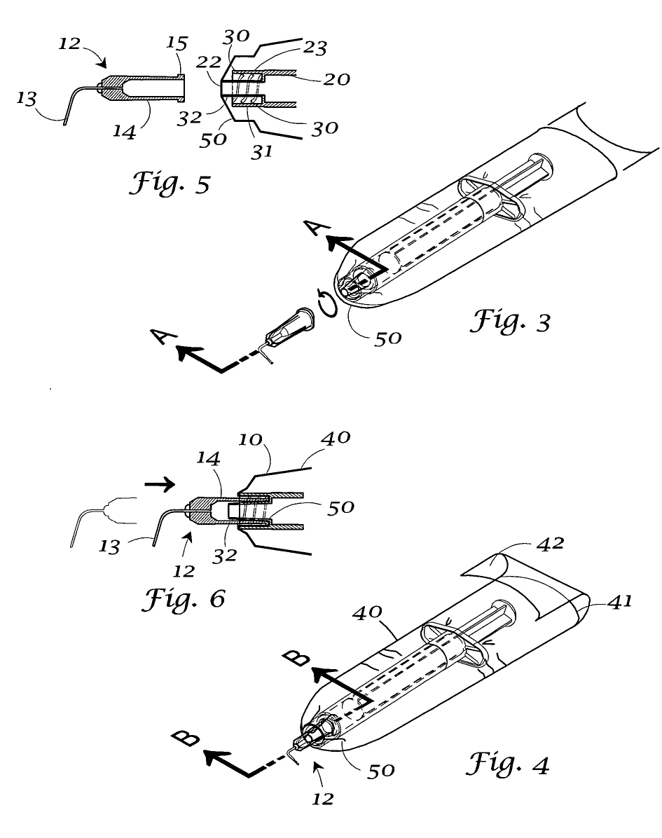

At the end of sleeve 10 opposite open end 41, is a syringe engaging section 50. Preferably, end 50 is solid, without an opening, but may be provided with an opening such as opening 51 shown in FIGS. 1 and 2. Syringe 11 is placed into sleeve 10 in the manner above described. End 22 of syringe 11 is caused to physically contact the interior of sleeve 10 at its end 50. It may be that dispensing protrusion 32 of syringe 11 engages the extreme end 50 of sleeve 10, if protrusion 32 is long enough, which is preferred but not required. It is to be appreciated that dispensing tip 12 is not in place upon syringe 10 (FIG. 3).

Dispensing tip 12 is then placed onto syringe 11 in its conventional manner, with the exception that end 50 of sleeve 10 is physically impinged between dispensing tip 12 and syringe 11 (FIG. 6). As shown in FIG. 6, a portion of end 50 of sleeve 10 is physically impinged between both body 14 of tip 12 and connector flange 15, and syringe 11. Further still, end 50 is pierced, broken or otherwise caused to open by this physical impingement, and in fact, when protrusion 32 is employed on syringe 11, actually forms around and even further seals protrusion 32. Material in syringe 11 can then be dispensed as normal through protrusion 11 and dispensing tip 12.

It will be appreciated that by reversing the procedure, the tip 12 can be removed from syringe 11 and syringe 11 can be removed from sleeve 10 without ever having come into direct contact with the operative environment. It is expected that each sleeve 10 will be used for only one patient and then discarded.

Sleeve 10 should be made, or at least its operative end 50 should be made, from a material thin enough to allow connection between tip 12 and syringe 11 in their intended manner.

It is to be appreciated, therefore, that a protective sleeve for a syringe is provided and accomplishes the objects of this invention, and otherwise provides an advancement and a contribution to the art.

We claim:

1. In combination, a material dispensing syringe having a replaceable tip, and a protective sheath for the syringe, comprising an elongated, flexible plastic sheath body with a syringe receiving opening at one end, and a syringe engaging end at the opposite end of said elongated body, wherein prior to placement of the tip onto the syringe, the syringe is receivable within said sheath by being passed through said syringe receiving opening; and wherein when the tip is placed onto the syringe, at least a portion of the sheath is physically impinged between the tip and the syringe.

2. A method of covering a replaceable tip, material dispensing syringe with a protective sheath, comprising the steps of inserting the syringe into a syringe receiving opening in the sheath and then placing the tip onto the syringe such that at least a portion of the sheath is physically impinged between the tip and the sheath.
